(19) United States
(12) Patent Application Publication
(10) Pub. No.: US 2009/0171176 A1
(43) Pub. Date: Jul. 2, 2009

(54) Snapshot Sensor

(75) Inventor: Lutz Andersohn, Glencoe, MO (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/343,742

(22) Filed: Dec. 24, 2008

Related U.S. Application Data
(60) Provisional application No. 61/009,451, filed on Dec. 28, 2007.

(51) Int. Cl. A61B 5/1455

(52) U.S. Cl. 600/324

(57) ABSTRACT

According to embodiments, there is provided a non-invasive medical device and method for using the same. Specifically, there is provided a pulse oximetry system that includes a sensor configured to detect electromagnetic radiation which has passed through living tissue and a monitor coupled to the sensor for processing information collected by the sensor. An actuation device is provided that is remotely located from the monitor and communicatively coupled to the monitor, wherein the monitor is configured to take a snapshot of physiological parameters and relay the physiological parameters to an electronic medical record (EMR) in response to receiving an actuation signal from the actuation device.

### RELATED APPLICATIONS

This application claims priority to U.S. Provisional Application No. 61/009,451, filed Dec. 28, 2007, and is incorporated herein by reference in its entirety.

### BACKGROUND

The present disclosure relates generally to medical devices and, more particularly, to sensors used with medical devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Conventional medical devices generally include sensors and monitors for collecting data and computing physiological parameters. Certain medical devices may also include actuation elements (e.g., push buttons) positioned on the monitors for activating the sensors to make instantaneous measurements. The act of activation is typically referred to as “taking a snapshot” as the data collected only reflects the measurement at the moment of activation. The medical devices configured to take snapshots may be configured to display and/or hold computed physiological parameters with data collected during the snapshot. Additionally, the data collected from a snapshot and/or the computed physiological data may be included in an electronic medical record (EMR).

EMRs are increasingly prevalent in the health care industry and are gradually supplanting the use of paper-based medical records. EMRs permit accurate exchanges of medical data among distinct information technology systems. The development of EMR interoperability standards is a primary objective of the national health care agenda. Additionally, EMR systems provide solutions to common problems related to paper-based records such as, for example, paper-based records not being easily transferred from one health care provider to another. Caregivers often rely on a patient's medical history reflected in medical records. If the records are not transferred, they may not be able to make an accurate diagnosis and duplicative testing may be performed. An EMR system can, therefore, provide increased accessibility, greater efficiency and improved patient care.

### BRIEF DESCRIPTION OF THE DRAWINGS

Certain embodiments are described in the following detailed description and in reference to the drawings in which:

FIG. 1 illustrates a simplified block diagram of a non-invasive medical device having an actuation device located on the sensor in accordance with an embodiment;

FIG. 2 illustrates a perspective view of a non-invasive medical device of FIG. 1 in accordance with an embodiment;

FIG. 3 illustrates a block diagram of an EMR system in accordance with an embodiment;

FIG. 4 is a flowchart illustrating operation of a medical device with an actuation device in accordance with an embodiment;

FIG. 5A illustrates a sensor having an actuation device located on a top surface of the sensor in accordance with an embodiment;

FIG. 5B illustrates a sensor having an actuation device located on a side surface of the sensor in accordance with an embodiment; and

FIG. 6 illustrated a snapshot actuation device being independent from a sensor and a monitor in accordance with an embodiment.

### SUMMARY

Certain aspects commensurate in scope with the disclosure are set forth below. It should be understood that these aspects are presented merely to provide the reader with a brief summary of certain forms the disclosure might take and that these aspects are not intended to limit the scope of the disclosure. Indeed, the disclosure may encompass a variety of aspects that may not be set forth below.

In accordance with one aspect of the present disclosure, there is provided a pulse oximetry system. The pulse oximetry system includes a sensor configured to detect electromagnetic radiation from a tissue site and a monitor operably coupled to the sensor and configured to process information collected by the sensor. The pulse oximetry system also includes an actuation device remotely located from the monitor and communicatively coupled to the monitor. The monitor is configured to take a snapshot of physiological parameters and relay the physiological parameters to an electronic medical record (EMR) in response to receiving an actuation signal from the actuation device.

In accordance with another aspect of the present disclosure, there is provided an electronic medical record (EMR) system. The EMR system includes an electronic media storage system and a medical device for determining physiological parameters coupled to the electronic media storage system. The medical device includes a sensor configured to detect a physiological signal of a patient, a monitor operably coupled to the sensor and configured to compute a physiological parameter based on the physiological signal received from the sensor. The medical device also includes an actuation device communicatively coupled to the monitor, wherein the monitor is configured to relay the computed physiological parameter to the electronic media storage system in response to receiving a signal from the actuation device.

Yet another aspect of the present disclosure provides a method of operating an EMR system. The method includes computing a physiological parameter using a monitor and taking a snapshot of the physiological parameter in response to detecting actuation of an actuation device located remotely from the monitor. An entry is made in a database of the physiological parameters of the snapshot.

In accordance with yet another aspect of the present disclosure, there is provided a sensor for use with a non-invasive medical device comprising an actuation device configured to prompt entry of data into an EMR upon actuation.

Another aspect of the present disclosure includes a pulse oximetry system comprising a sensor configured to detect electromagnetic radiation from a tissue site and a monitor operably coupled to the sensor and configured to process information collected by the sensor. The pulse oximetry system also includes an actuation device located on the sensor and communicatively coupled to the monitor, wherein the monitor is configured to take a snapshot of physiological parameters in response to receiving an actuation signal from the actuation device.

In accordance with yet another aspect of the present disclosure, there is provided a method of operating a medical device. The method includes computing a physiological parameter using a monitor and taking a snapshot of the physiological parameter in response to detecting actuation of an actuation device located on a sensor operably coupled to the monitor.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

An uncooperative patient may make the task of obtaining accurate measurements using conventional medical devices difficult. For instance, a caregiver may need to use both hands to steady a sensor of a non-invasive medical device on the finger of an uncooperative patient thus making it difficult to activate the sensor by pressing an actuation element located on a monitor portion of the non-invasive medical device. In accordance with the present disclosure, a medical device and a method of operating the medical device having an actuation device which may be located remotely from a monitor are provided. Because the actuation device is located independently from the monitor, a caregiver may more easily activate a sensor of the medical device while positioning the sensor upon a patient.

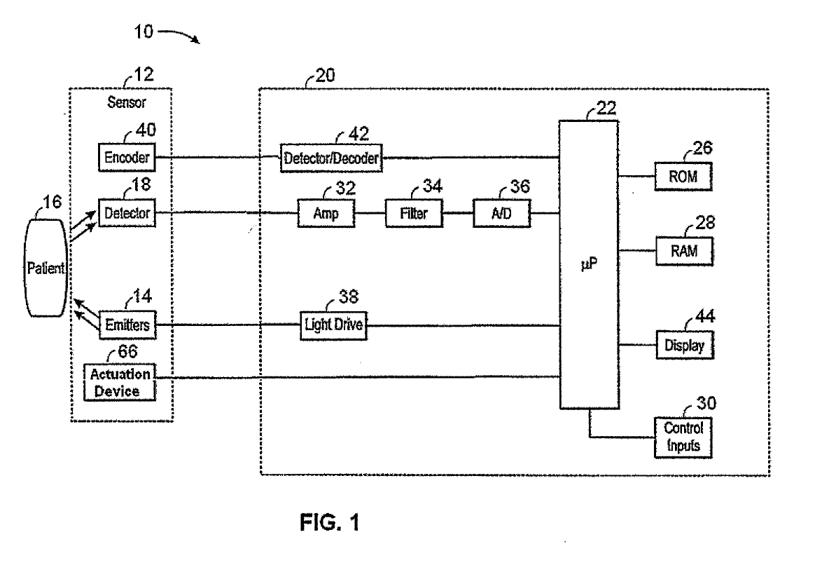

Turning to FIG. 1, a block diagram of a non-invasive medical device, such as a pulse oximeter, for example, is illustrated in accordance with an exemplary embodiment and is generally designated with the reference number 10. For example, the device 10 may be an oximeter available from Nellcor Puritan Bennett L.L.C. The non-invasive medical device 10 may include a sensor 12 having an emitter 14 configured to transmit electromagnetic radiation, i.e., light, into the tissue of a patient 16. The emitter 14 may include a plurality of LEDs operating at discrete wavelengths, such as in the red and infrared portions of the electromagnetic radiation spectrum for example. Alternatively, the emitter 14 may be a broad spectrum emitter.

A photoelectric detector 18 in the sensor 12 may be configured to detect the scattered and/or reflected light from the tissue and to generate an electrical signal, e.g., current, corresponding to the detected light. The sensor 12 may direct a detected signal from the detector 18 to a monitor 20 that processes the signal and calculates physiological parameters.

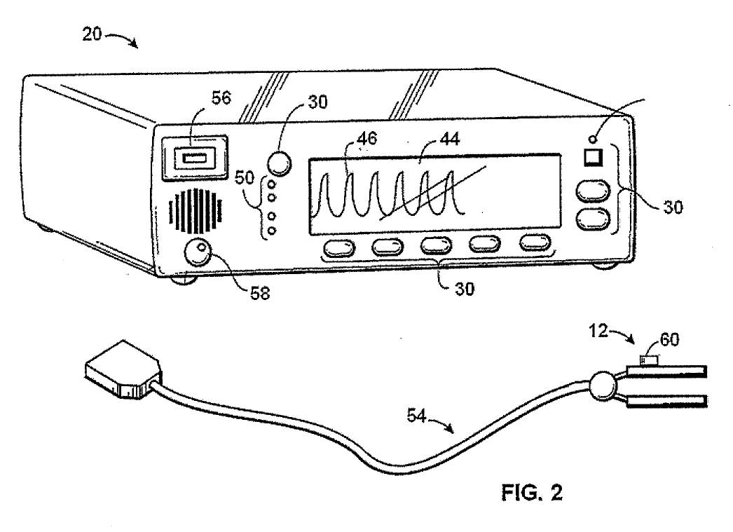

The monitor 20 may include a microprocessor 22 configured to calculate physiological parameters using algorithms programmed into the monitor 20. The microprocessor 22 may be connected to other component parts of the monitor 20, such as a ROM 26, a RAM 28, and control inputs 30. The ROM 26 may be configured to store the algorithms used to compute physiological parameters. The RAM 28 may store the values detected by the detector 18 for use in the algorithms. The inputs 30 may allow a user, such as a clinician, for example, to interface with the monitor 20. Specifically, as will be described in greater detail with regard to FIG. 2 below, the control inputs 30 may allow for a clinician to scroll through screens of historical data and/or select items from a menu.

The monitor 20 may amplify and filter the signals using an amplifier 32 and a filter 34, respectively, before an analog-to-digital converter 36 digitizes the signals. Once digitized, the signals maybe used to calculate the physiological parameters and/or may be stored in the RAM 28.

A light drive unit 38 in the monitor 20 may control the timing of the emitters 14. While the emitters 14 may be manufactured to operate at one or more discrete wavelengths, variances in the wavelengths actually emitted may occur. As such, an encoder 40 and decoder 42 may be used to calibrate the monitor 20 to the actual wavelengths being used. The encoder 40 may be a resistor, for example, whose value corresponds to the actual wavelengths and to coefficients used in algorithms for computing the physiological parameters. Alternatively, the encoder 40 may be a memory device, such as an EPROM, that stores wavelength information and/or the corresponding coefficients. Once the coefficients are determined by the monitor 20, they may be inserted into the algorithms in order to calibrate the pulse oximeter 10.

The monitor 20 may be configured to display the calculated parameters on a display 44. As illustrated in FIG. 2A, the display 44 may be integrated into the monitor 20. However, in an embodiment, the monitor 20 may be configured to provide data via a port to a display (not shown) that is not integrated with the monitor 20. The display 44 may be configured to display computed physiological data including, for example, a percent oxygen saturation, a pulse rate and/or a plethysmographic waveform 46. As is known in the art, the oxygen saturation may be a functional arterial hemoglobin oxygen saturation measurement in units of percentage SpO₂, and the pulse rate may indicate a patient's pulse rate in beats per minute. The monitor 20 may also display information related to alarms, monitor settings, and/or signal quality via indicator lights 50.

To facilitate user input, the monitor 20 may include control inputs 30 of FIG. 1. The control inputs may include fixed function keys, programmable function keys, and soft keys. Specifically, the control inputs 30 may correspond to soft key icons in the display 44. Pressing control inputs 30 associated with, or adjacent to, an icon in the display selects a corresponding option.

The sensor 12 may be communicatively coupled to the monitor 20 via a cable 54 which connects to a sensor port 56 on the monitor 20. As mentioned above, an actuation device, such as button 58, for example, may be provided on the monitor 20 which, when activated, may cause the monitor 20 to take a snapshot of current physiological parameters. However, in accordance with an embodiment, an actuation device 60 may be provided independent from the monitor 20 to provide remote actuation. FIGS. 1 and 2 each illustrate an embodiment wherein an actuation device 60 may be provided on the sensor 12. The positioning of the actuation device 60 remotely from the monitor 20 can improve a caregiver's access to the actuation device 60. For instance, a caregiver may be able to activate the actuation device 60 while using both hands to properly position the sensor 12 upon an uncooperative patient, for instance.

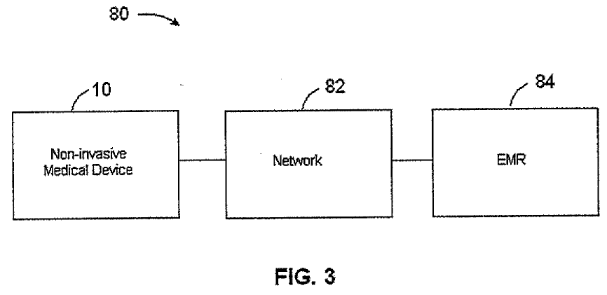

The actuation device 60 may be any appropriate actuation device, such as a push button switch, for example, that when pressed sends a signal to the monitor 20 indicating that the monitor 20 should take a snapshot. Additionally, the actuation of the actuation device 60 may indicate that the data associated with the snapshot, such as any computed physiological data, and even perhaps the raw data in digital form, be provided to an electronic medial record (EMR). FIG. 3 illustrates a block diagram of an EMR system 80 in accordance with an exemplary embodiment. As can be seen, the EMR system 80 may include a network 82 which is coupled to the non-invasive medical device 10. The system also includes an EMR 84 which is coupled to the network 82. The network 82 may include routers, wireless base stations, and server computers, among other things. The network 82 may be any suitable network, such as a local area network, campus area network, a metropolitan area network, or a wide area network depending, on the desired use of the EMR system 80. For example, the network 82 may be used in a single hospital, a single floor of a hospital, a hospital network, or hospitals and health centers within a city or state. However, because of the sensitive nature of the information contained in the EMR 84, appropriate security measures may be deployed to restrict access to the information.

The EMR system 84 may include an electronic storage device or a plurality of memory devices configured to operate as a database for storing patient information including the physiological parameters collected by the non-invasive medical device 10. For example, the EMR 84 may include a storage area network coupled to the servers of the network 82, or alternatively may simply include a hard drive device that may be integrated into a server of the network 82, depending on the intended volume of data that will be stored by the EMR 84. In one exemplary embodiment, the EMR 84 may be located in a separate building, city, or state, from the non-invasive medical device 10. In the configuration illustrated, the non-invasive medical device 10 may make entries directly into the EMR 84 via the network 82, thus eliminating the need for evaluation of a patient by a caregiver and subsequent data entry into the EMR 84. Additionally, the non-invasive medical device 10 may be configured to retrieve data stored in the EMR 84 to allow a caregiver to review a patient's medical history and/or trend data while with the patient.

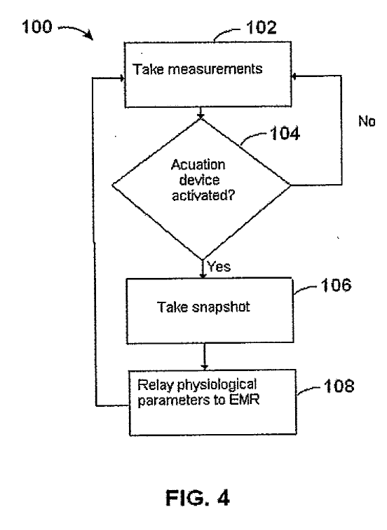

Referring to FIG. 4, a flow chart illustrates a manner in which a medical device 10, such as a non-invasive medical device, having an actuation device 60 independent from the monitor 20 may be operated. The monitor 20 may be configured to take continuous measurements (block 102). The monitor 20 may be configured to compute, display and store physiological parameters while taking continuous measurements. Alternatively, the monitor 20 may be configured to simply take measurements without computing physiological parameters. The monitor 20 may be configured to periodically poll for an interrupt signal originating from the actuation device 60 indicating that the actuation device 60 has been activated (block 104). If the actuation device 60 has not been activated, the monitor 20 continues to take measurements. However, once the activation device 60 is activated, the monitor 20 takes a snapshot (block 106). The taking of the snapshot may include computing physiological data for the instant that the actuation device 60 was activated and freezing the computed data displayed on the display 44 to allow a caregiver to evaluate the data. The monitor 20 may be configured to store snapshots for review on the display 44 by a caregiver. The stored snapshots may be deleted, saved or even relayed to an EMR at some point. Indeed, the activation of the actuation device 60 may cause the non-invasive medical device 10 to make an entry in an EMR 84 (block 108). After the snapshot has been taken and/or information has been relayed to the EMR 84, the monitor 20 return to continuous measurements. Alternatively, the monitor 20 may not be configured to take continuous measurements and may, instead, take a measurement only when the actuation device 60 is activated.

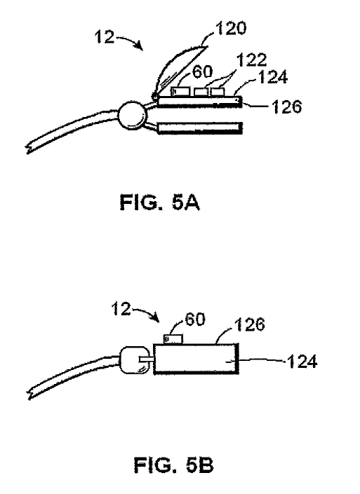

A cover 120 may be provided to protect against inadvertent actuation of the actuation device 60, as illustrated in FIG. 5A. The cover 120 may be attached to the sensor 12 by a hinge that allows for the cover 120 to pivot away form the sensor 12. Additionally, as illustrated, additional buttons 122 may be provided on the sensor 12 to increase convenience and functionality. For example, buttons 122 may be configured to scroll through and/or select menu items displayed on the display 44, for example. In particular, in accordance with one embodiment, the additional buttons 122 may be configured to allow for a caregiver to indicate that a particular snapshot was a good snapshot based on physiological data displayed on the display 44. In such a configuration, the monitor 14 may be configured to relay a snapshot to the EMR 84 only after it has been indicated as being a good snapshot, thus helping to prevent filling the EMR 84 with data that is not useful. The additional buttons 122 may be located adjacent to the actuation device 60 on a top surface 124 of the sensor 12 and may also be covered by the cover 120.

In an embodiment, the actuation device 60 may not be located on the top surface 124 of the sensor 12. In particular, as illustrated in FIG. 5B, the actuation device 60 may be located on a side surface 126 of the sensor 12. This embodiment may additionally help to avoid inadvertent actuation of the actuation device 60. Similar to the embodiment shown in FIG. 5A, a cover (not shown) and/or addition buttons (not shown) may be provided with the actuation button 60 located on the side surface 126 of the sensor 12.

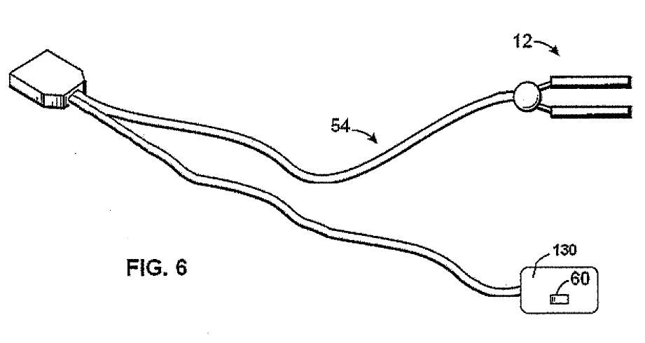

In yet another embodiment, the actuation device 60 may be independent from both the sensor 12 and the monitor 20, as illustrated in FIG. 6. Specifically, the actuation device 60 may be located on a device 130, which may be a handheld device such as a remote control or, alternatively, a foot pedal that may be positioned on the floor under a chair or bed where the patient is located, for example. Thus, the caregiver may activate the actuation device without leaving the patient and/or while holding the sensor 12 in place. Additionally, the device 130 may be configured to communicate with the monitor 14 wirelessly.

As discussed in detail above, in addition to taking measurements upon actuation of the actuation device 60, the monitor 20 may be configured to provide snapshot data automatically to an EMR 84, possibly saving a significant amount of time for caregivers. Because the actuation device is conveniently located remotely from the monitor 12, such as on the sensor 12, for example, a caregiver may activate the actuation device 60 while holding the sensor 12 in place to obtain accurate readings.

While the disclosure may be susceptible to various modifications and alternative forms, specific embodiments have been shown by way of example in the drawings and have been described in detail herein. However, it should be understood that the disclosure is not intended to be limited to the particular forms disclosed. Rather, the disclosure is to cover all modifications, equivalents, and alternatives falling within the spirit and scope of the disclosure as defined by the following appended claims.

What is claimed is:

1. A pulse oximetry system, comprising:
a sensor configured to detect electromagnetic radiation from a tissue site;
a monitor operably coupled to the sensor and configured to process information collected by the sensor; and
an actuation device remotely located from the monitor and communicatively coupled to the monitor, wherein the monitor is configured to take a snapshot of physiological parameters and relay the physiological parameters to an electronic medical record (EMR) in response to receiving an actuation signal from the actuation device.

2. The pulse oximetry system of claim 1, wherein the actuation device is located on the sensor.

3. The pulse oximetry system of claim 2, wherein the sensor comprises a cover generally positioned over the actuation device.

4. The pulse oximetry system of claim 1, wherein the actuation device comprises a foot switch.

5. The pulse oximetry system of claim 2, wherein the actuation device comprises a push button switch.

6. The pulse oximetry system of claim 2, wherein the sensor comprises control inputs for operating the monitor,

7. An electronic medical record (EMR) system, comprising:
an electronic media storage system; and
a medical device for determining physiological parameters coupled to the electronic media storage system, the medical device comprising:
a sensor configured to detect a physiological signal of a patient;
a monitor operably coupled to the sensor, the monitor configured to compute a physiological parameter based at least in part upon the physiological signal received from the sensor; and
an actuation device communicatively coupled to the monitor, wherein the monitor is configured to relay the computed physiological parameter to the electronic media storage system in response to receiving a signal from the actuation device.

8. The EMR system of claim 7, comprising a computer network for coupling the medical device to the electronic media storage system.

9. The EMR system of claim 7, wherein the actuation device is located on the sensor.

10. The EMR system of claim 9, wherein the actuation device comprises a push button.

11. The EMR system of claim 10, wherein the sensor comprises a cover positioned over the push button.

12. The EMR system of claim 7, wherein the actuation device comprises a foot switch.

13. The EMR system of claim 9, wherein the monitor comprises control inputs on the sensor.

14. A method of operating an EMR system, comprising:
computing a physiological parameter using a monitor;
taking a snapshot of the physiological parameter in response to detecting actuation of an actuation device located remotely from the monitor; and
making an entry in a database of the physiological parameters of the physiological parameter of the snapshot.

15. The method of claim 14, wherein making an entry in the database comprises providing the physiological parameter to the database via a network.

16. The method of claim 14, wherein computing a physiological parameter comprises computing a pulse rate.

17. The method of claim 14, wherein computing a physiological parameter comprises computing percent oxygen saturation of hemoglobin.

18. A sensor for use with a non-invasive medical device comprising an actuation device configured to prompt entry of data into an EMR upon actuation.

19. The sensor of claim 18 comprising a hinged cover configured to cover the actuation device.

20. The sensor of claim 18, wherein the actuation device is located on a side surface of the sensor.
